(19) United States
(12) Patent Application Publication
(10) Pub. No.: US 2018/0133424 A1
(43) Pub. Date: May 17, 2018

(54) ENDOTRACHEAL TUBE AND METHOD OF USE THEREOF

(71) Applicant: COOK MEDICAL TECHNOLOGIES LLC, BLOOMINGTON, IN (US)

(72) Inventor: Steven L. Pendleton, Spencer, IN (US)

(21) Appl. No.: 15/785,733

(22) Filed: Oct. 17, 2017

Related U.S. Application Data
(60) Provisional application No. 62/422,133, filed on Nov. 15, 2016.

(51) Int. Cl. A61M 16/04; A61L 29/08; A61L 29/06

(52) U.S. Cl. CPC A61M 16/0443; A61M 16/0445; A61M 2025/1075; A61L 29/085; A61L 29/06; A61M 16/044

(57) ABSTRACT

The present disclosure relates generally to an endotracheal tube and to a method of using such a device. One embodiment of the device includes an inflatable balloon having at least a portion of its surface covered with a super-hydrophobic coating. In another embodiment, the inflatable balloon has a variable wall thickness which can allow for expansion of the balloon against the oropharynx to prevent tissue pressure necrosis within the trachea.

### RELATED APPLICATIONS

The present application claims priority from U.S. Provisional Patent Application No. 62/422,133, filed Nov. 15, 2016, the contents of which are hereby incorporated by reference in their entirety.

### TECHNICAL FIELD

The present disclosure relates generally to an endotracheal tube and to a method of using such a device. One embodiment of the device includes an inflatable balloon having at least a portion of its surface covered with a super-hydrophobic coating. In another embodiment, the inflatable balloon has a variable wall thickness allowing for expansion of the balloon against the oropharynx to prevent tissue pressure necrosis within the trachea.

### BACKGROUND

Ventilation is a physiologic process which supplies oxygen to the body and removes carbon dioxide, a gaseous waste product. Ventilation is provided by the rhythmic back and forth motion of air in the trachea, caused by the rhythmic contraction and relaxation of the diaphragm. In seriously ill or injured patients unable to breathe adequately on their own, ventilation can be assisted by inserting an endotracheal tube through the oral or nasal cavity of the patient, a process often referred to as endotracheal intubation. An endotracheal tube is a single or double-lumen catheter that is open at both ends. One end extends outside of the patient and is engaged with a mechanical ventilator for supplying a ventilation fluid. The other end extends between the vocal cords and into the trachea of the patient.

Proper placement of the endotracheal tube typically requires the use of a guide instrument, such as a laryngoscope or a video-laryngoscope, to provide a degree of visualization of the internal anatomy of the patient. The laryngoscope may include a curved blade-like structure that is inserted into the pharynx. The blade-like structure elevates the epiglottis to provide a view of the vocal cords and the glottis, and provides a pathway for the end of the endotracheal tube to be manually directed past the vocal cords, and into the trachea. A handle engaged with the blade extends outside the throat to facilitate manipulation by the medical professional. During the intubation procedure, the professional typically grasps the handle of the laryngoscope with one hand, and controls the position of the endotracheal tube with the other hand.

A conventional cuffed tracheal tube for establishing an airway in a patient is intubated into the trachea of the patient through the mouth, nose or a tracheostomy stoma of the patient. The tracheal tube includes an inflatable cuff which is inflatable to seal against the wall of the trachea. However, secretions derived from saliva and mucus in the trachea may collect above the cuff and may cause infections. A cuffed tracheal tube may have a suction opening disposed above the inflatable cuff for suctioning the secretions collecting above the cuff so as to reduce the risk of chest infections in long-term intubated patients. However, when a vacuum is applied to suction the secretions through the suction opening, the vacuum may cause the tracheal wall mucosa membrane to be drawn into the suction opening, which may result in injury to the tracheal wall and obstruct removal of the secretions.

In addition, incomplete sealing by the balloon against the tracheal wall may result in fluid entering the lungs, resulting in ventilator-associated pneumonia. Attempts to avoid such a condition by over-inflation of the inflation cuff may result in tissue damage to the tracheal wall.

### SUMMARY

In one aspect, the present invention provides a tracheal device including an elongated member having a lumen extending therethrough from a proximal end to a distal end. An inflatable balloon attaches to the elongated member. The balloon includes an inflation port and an inflation tube extending from the inflation port towards the proximal end and in fluid contact with the interior of the inflatable balloon. The inflatable balloon includes a proximal end having a wall thickness less than a wall thickness of the distal end of the balloon.

In one embodiment, the external surface of the balloon includes a hydrophobic coating. The coating may include a hydrophobic polymer. The inflatable balloon may be of a size and shape to block a human trachea when positioned at a top of the trachea and inflated to contact the wall of the trachea. In one embodiment, further inflation of the balloon after the balloon abuts the wall of the trachea is accommodated by proximal expansion of the balloon portion above an opening of the trachea.

In another embodiment, the device includes a flap having a first end attached to the elongated member proximally of the inflatable balloon and extending distally from the first end to a free end. The surface of the flap may include a super-hydrophobic coating.

The inflatable balloon may include a silicone. In one embodiment, the balloon proximal end has a greater lateral dimension than that of the balloon distal end. In another embodiment, the wall thickness at the balloon proximal end is less than 50% of that of the wall thickness at the balloon distal end. In yet another embodiment, the wall thickness at the balloon proximal end is less than 30% of that of the wall thickness at the balloon distal end.

In another embodiment, the exterior surface of the balloon is coated with a super-hydrophobic material. The inside wall of the lumen of the elongated member may also be coated with a super-hydrophobic material. In one embodiment, the super-hydrophobic material is selected from the group consisting of manganese oxide polystyrene (MnO2/PS) nano-composite, zinc oxide polystyrene (ZnO/PS) nano-composite, precipitated calcium carbonate, a carbon nano-tube structure and a silica nano-coating.

In one embodiment, the proximal portion of the balloon is a compliant portion and the distal portion of the balloon is a non-compliant portion.

### BRIEF DESCRIPTION OF THE DRAWINGS

FIG. 1 is an illustration of one embodiment of a tracheal device of the present invention

FIG. 2 is another illustration of the tracheal device of FIG. 1.

FIG. 3 is an illustration of another embodiment of the tracheal device of the present invention.

FIG. 4 is another illustration of the tracheal device of FIG. 3.

### DETAILED DESCRIPTION OF THE PREFERRED EMBODIMENTS

### Definitions

For purposes of promoting an understanding of the present invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the intubation device, as well as the axial ends of various component features. The term “proximal” is used in its conventional sense to refer to the end of the assembly (or component) that is closest to the medical professional during use of the assembly. The term “distal” is used in its conventional sense to refer to the end of the assembly (or component) that is initially inserted into the patient, or that is closest to the patient during use. The term “longitudinal” will be used to refer to an axis that aligns with the proximal-distal axis of the device. The term “lateral” will be used to refer to an axis or plane that is perpendicular to the proximal-distal axis of the device.

The uses of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein the terms “comprise(s),” “include(s),” “having,” “has,” “can,” “contain(s),” and variants thereof, are intended to be open-ended transitional phrases, terms, or words that do not preclude the possibility of additional acts or structures. The present invention also contemplates other embodiments “comprising,” “consisting of” and “consisting essentially of,” the embodiments or elements presented herein, whether explicitly set forth or not.

As used herein the term hydrophobic coating refers to a coating on which the contact angle of a water droplet exceeds 90° (e.g., ranging from 90° to 100° or to 120° or to 140° or to 145°).

As used herein the term super-hydrophobic coating refers to a coating on which the contact angle of a water droplet exceeds 150° (e.g., ranging from 150° to 155° or to 160° or to 165° or to 170° or to 175° to 180°). In some embodiments the roll-off angle/contact angle hysteresis is less than 10°.

### Endotracheal Device

One aspect of the present invention provides an endotracheal tube (ETT) device. The device includes an inflatable balloon cuff located on an endotracheal tube shaft. The balloon cuff is sized and shaped to custom fit all sizes of tracheal openings and to butt firmly against the oropharynx to create a water-slide that diverts the mouth secretions/saliva away from the trachea and into the esophagus. In certain embodiments, the device may also include a flap that tucks into the esophagus and directs secretion/saliva from the tracheal opening.

In one embodiment, the balloon cuff provides a custom fit to the anatomy of the tracheal entry region by having a variable wall thicknesses that allows the clinician to fill the balloon with the required amount of fluid (for example, sterile saline) and that causes the balloon to expand to plug the top of the trachea and abut against the bottom of the oropharynx. In another embodiment, the balloon has a shape and wall thickness that causes the balloon, when over-inflated, to abut against the oropharynx so that any pressure caused by the over inflation is relieved into the Adam's apple area of the throat. Such a construction prevents tissue pressure necrosis from occurring within the trachea.

The balloon may be formed from a polymeric material. For example, the balloon material may include at least one of polyethylene teraphthalate (PETP), low-density polyethylene (LDPE), polyvinyl chloride (PVC), silicone, neoprene, polyisoprene, polypropylene, or polyurethane (PU). In a preferred embodiment, the balloon is formed of silicone.

The device, and in particular, the balloon, may have a coating including a water repellent hydrophobic or super-hydrophobic material. When saliva/secretions saliva touch the coating, this material slides/bounces away from the surface and into the esophagus, thus keeping the material out of the trachea and lungs.

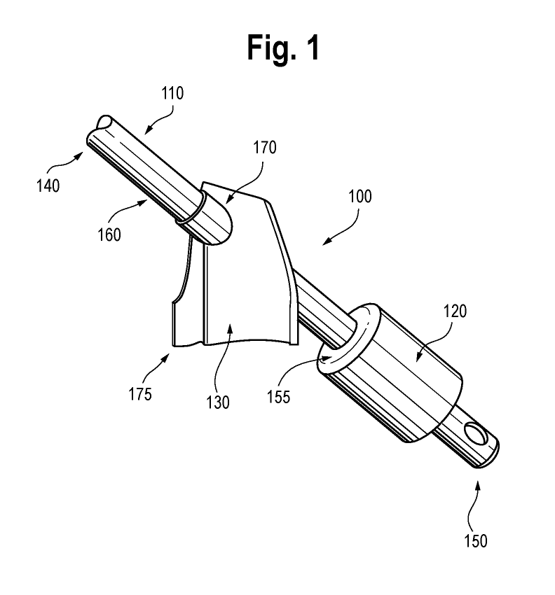

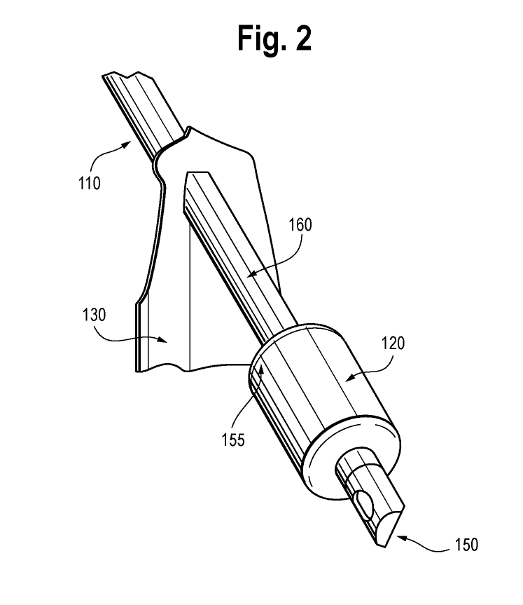

FIGS. 1 and 2 show two views of one embodiment of an endotracheal device of the present invention. Endotracheal device 100 includes elongated member 110 having a lumen extending therethrough from proximal end 140 to distal end 150. Inflatable balloon 120 attaches to elongated member 110 and includes inflation port 155. Inflation tube 160 extends from inflation port 155 towards proximal end 140 and is in fluid contact with the interior of the inflatable balloon.

In one embodiment, the elongated member extends through the inflatable balloon. In another embodiment, the inflatable balloon attached to a side of the elongated member and expands abound the elongated member when inflated. In both of these embodiments, when inflated, the balloon seals the entrance to the trachea around the exterior of the elongated member such that fluid may be supplied through the lumen of the elongated member whereas secretions are prevented from entering the trachea from outside of the lumen of the elongated member.

The external surface of balloon 120 may include a hydrophobic or super-hydrophobic coating. For example, the entire surface of the balloon may have such a coating. In other embodiments, only part of the exterior surface of the balloon, for example the proximal portion, may be coated. In one embodiment, the hydrophobic coating includes a hydrophobic polymer.

In a preferred embodiment, the balloon, or part of the balloon, includes a super-hydrophobic coating. For example, the super-hydrophobic coating may include a material such as manganese oxide polystyrene (MnO2/PS) nano-composite, zinc oxide polystyrene (ZnO/PS) nano-composite, precipitated calcium carbonate, a carbon nano-tube structure, a silica nano-coating, or a combination of at least two of these materials. Methods of forming super-hydrophobic coatings are known to those skilled in the art. Examples of such methods are described, for example, in US Publication Number 2007/0005024A1, published Jan. 4, 2007, the contents of which are incorporated by reference.

The interior of elongated member 110 may also be coated with such a hydrophobic or super-hydrophobic material. Preferably, the interior of the elongated member is coated with a super-hydrophobic material. Such a coating offers two advantages. First, it prevents mucous plugs from plugging the lumen of the elongated member. With conventional endotracheal tubes, such plugging occurs in about 1% of all tracheostomy tubes and can lead to death of the patient by suffocation. Furthermore, the coated prevents lumen narrowing caused by biofilm accumulation. Such narrowing may lead to ventilation inefficiency, which may require clinician intervention to remove the biofilm from the lumen.

In one embodiment, endotracheal device 100 includes flap 130. The presence of such a flap may reduce or eliminate the need for the suctioning of secretions during intubation. First end 170 of flap 130 attaches to a portion of elongated member 110 proximally of balloon 120. Flap 130 extends distally from the attachment point to free end 175. When the device is positioned in the trachea of a patent, flap 130 covers the trachea opening and diverts flow of mouth secretions/saliva into the esophagus.

Flap 130 may be formed from a soft polymer material (e.g. silicone) and may freely rotate on elongated member 110. When positioned in the trachea, the free end may be affixed to the epiglottis by hanging or bioadhesive gluing to provide the flap longitudinal position stability (i.e. so that it does not fall into the esophagus); but keeping a horizontal motion free from the elongated member so as to prevent epiglottis tissue damage during patient/device movement. The surface of flap 130 may include a hydrophobic or super-hydrophobic coating such as those disclosed herein. Of course, the present embodiments also include endotracheal devices not including a flap as disclosed herein.

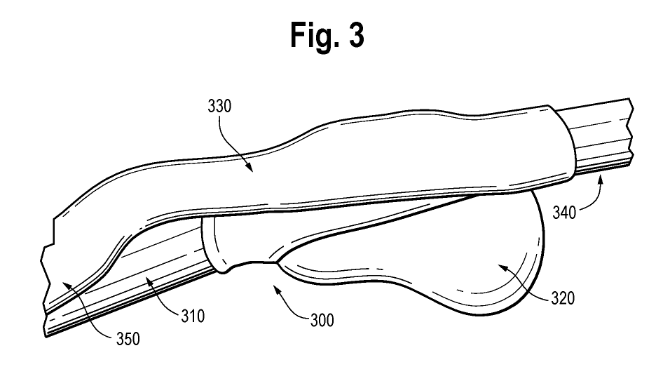

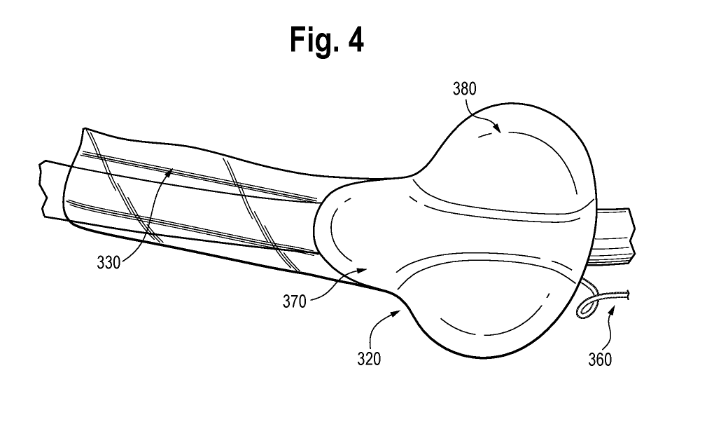

Turning now to FIGS. 3 and 4, where is here shown two views of another embodiment of an endotracheal device of the present invention. Endotracheal device 300 includes elongated member 310 having a distal end 350 and a proximal end 340. Inflatable balloon 320 attaches to elongated member 310 and includes an inflation tube 360 extending from an inflation port and towards proximal end 340. For example, balloon 320 may attach to a central or a proximal portion of elongated member 320. In any case, balloon 320 is attached such that, when the device is positioned within the patient, the balloon is positioned at the opening to the trachea and seals the opening around elongated member 310. In some embodiments, endotracheal device 300 may also include flap 330. The flap serves the same function as flap 130 described herein.

Inflatable balloon 320 is sized and shaped to block a human trachea when positioned at a top of the trachea and inflated to contact a wall of the trachea. In one embodiment, balloon 320 includes a distal portion 370 having a wall that is thicker than the wall of the proximal portion 380 of the balloon. In this embodiment, inflation of the balloon causes both portions of the balloon to inflate until the distal portion contacts the wall of the trachea. Further inflation of the balloon is accommodated by expansion of the balloon proximal above the opening of the trachea.

As a result, pressure caused by the over inflation of the balloon is relieved into the Adam's apple area of the throat and tissue pressure necrosis is prevented within the trachea. In one embodiment, accommodation of over-inflation within the proximal region of the balloon is achieved by providing a balloon having a reduced wall thickness in the proximal region of the balloon. For example, a proximal portion of the balloon may have a wall thickness that is less than 80%, 70%, 60% 50%, 40%, 30% or 20% of a wall thickness at the balloon distal end.

In another embodiment, the balloon proximal end has a greater lateral dimension than that of the balloon distal end. For example, in the embodiment illustrated in FIGS. 3 and 4, proximal balloon portion 380 has a greater lateral dimension than distal balloon portion 370. In some embodiments, the proximal portion of the balloon is a compliant portion, whereas the distal portion of the balloon is a non-compliant portion. In such embodiments, the distal portion of the balloon, which is positioned within the trachea, will inflate to seal the entrance to the trachea. Over inflation of the balloon with be accommodated by expansion of the proximal portion.

Another aspect of the invention provides a method of intubating a subject with a device as disclosed herein. The method includes the step of inserting the distal end of the device into the trachea of the patient so as to position the inflatable balloon at the entrance to the trachea. The balloon is then inflated with a fluid until the distal portion of the balloon contacts the trachea wall to seal the trachea. Any over inflation of the balloon will be accommodated by additional expansion of the proximal portion of the balloon above the trachea entrance.

If the device includes a flap, the free end of the flap may be inserted into the esophagus to direct fluids away from the entrance to the trachea. In addition, a coating of a hydrophobic or super-hydrophobic material on the balloon, particularly on the proximal portion of the balloon, will cause any secretions that fall onto the balloon to be repelled from the surface and into the esophagus.

The balloon may be inflated with a gas or a liquid. In a preferred embodiment, the balloon is inflated with sterile saline. Inflating the balloon with a liquid offers advantages in that the balloon will not deflate over times, as is sometimes the case with air-filled balloons.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

What is claimed is:

1. A tracheal device comprising
an elongated member having a lumen extending therethrough from a proximal end to a distal end;
an inflatable balloon attaching to the elongated member and comprising an inflation port; and
an inflation tube extending from the inflation port towards the proximal end and in fluid contact with an interior of the inflatable balloon,
wherein the inflatable balloon comprises a balloon proximal end having a wall thickness less than a wall thickness of a balloon distal end.

2. The tracheal device of claim 1, wherein an external surface of the balloon proximal end comprises a hydrophobic coating.

3. The tracheal device of claim 2, wherein the hydrophobic coating comprises a hydrophobic polymer.

4. The tracheal device of claim 1, further comprising a flap having a first end attaching to the elongated member proximally of the inflatable balloon and extending distally from the first end to a free end.

5. The tracheal device of claim 4, wherein a surface of the flap comprises a super-hydrophobic coating.

6. The tracheal device of claim 1, wherein the inflatable balloon is sized and shaped to block an human trachea when positioned at a top of the trachea and inflated to contact a wall of the trachea.

7. The tracheal device of claim 6, wherein further inflation of the inflatable balloon after the inflatable balloon abuts the wall of the trachea is accommodated by proximal expansion of the balloon proximal above an opening of the trachea.

8. The tracheal device of claim 1, wherein the inflatable balloon comprises a silicone.

9. The tracheal device of claim 1, wherein a wall thickness at the balloon proximal end is less than 50% of that of a wall thickness at the balloon distal end.

10. The tracheal device of claim 9, wherein the wall thickness at the balloon proximal end is less than 30% of that of the wall thickness at the balloon distal end.

11. The tracheal device of claim 1, wherein the balloon proximal end has a greater lateral dimension than that of the balloon distal end.

12. The tracheal device of claim 1, wherein the balloon comprises a coating comprising a super-hydrophobic material.

13. The tracheal device of claim 12, wherein the super-hydrophobic material is selected from the group consisting of manganese oxide polystyrene (MnO2/PS) nano-composite, zinc oxide polystyrene (ZnO/PS) nano-composite, precipitated calcium carbonate, a carbon nano-tube structure and a silica nano-coating.

14. The tracheal device of claim 1, wherein a proximal portion of the balloon is a compliant portion and wherein a distal portion of the balloon is a non-compliant portion.

15. The tracheal device of claim 1, wherein an inside wall of the lumen comprises a coating comprising a super-hydrophobic material.

16. The tracheal device of claim 15, wherein the super-hydrophobic material is selected from the group consisting of manganese oxide polystyrene (MnO2/PS) nano-composite, zinc oxide polystyrene (ZnO/PS) nano-composite, precipitated calcium carbonate, a carbon nano-tube structure and a silica nano-coating.

17. The tracheal device of claim 1, wherein the inflatable balloon attaches to a central portion of the elongated member.

18. A tracheal device comprising
an elongated member having a lumen extending therethrough from a proximal end to a distal end, wherein an inside wall of the elongated member comprises a super-hydrophobic coating;
an inflatable balloon attaching to the elongated member and comprising an inflation port, wherein an exterior surface of the inflatable balloon comprises a super-hydrophobic coating; and
an inflation tube extending from the inflation port towards the proximal end and in fluid contact with an interior of the inflatable balloon,
wherein the inflatable balloon comprises a balloon proximal end having a wall thickness less than a wall thickness of a balloon distal end.

19. The tracheal device of claim 18, wherein the super-hydrophobic coating comprises a material selected from the group consisting of manganese oxide polystyrene (MnO2/PS) nano-composite, zinc oxide polystyrene (ZnO/PS) nano-composite, precipitated calcium carbonate, a carbon nano-tube structure and a silica nano-coating.
